(19) United States
(12) Patent Application Publication
(10) Pub. No.: US 2013/0022554 A1
(43) Pub. Date: Jan. 24, 2013

(54) Oral care formulations

(71) Applicants: Rebecca L. Engel, Kalamazoo, MI (US); David F. Felicelli, Holland, MI (US)

(72) Inventors: Rebecca L. Engel, Kalamazoo, MI (US); David F. Felicelli, Holland, MI (US)

(21) Appl. No.: 13/136,038

(22) Filed: Jul. 20, 2011

(51) Int. Cl. A61K 8/55; A61K 8/24; A61Q 11/00; A61K 8/44

(52) U.S. Cl. 424/53; 424/57; 424/54

(57) ABSTRACT

A formulation for an oral care application comprises a linear polyphosphate or a polyphosphate blend having a short average effective chain length. The polyphosphate blend may further comprise a cyclic phosphate and a linear polyphosphate. The formulation provides whitening effects, decreased tooth sensitivity, strengthened tooth enamel, and remineralization. The present invention further relates to methods for making an oral care formulation comprising a linear polyphosphate or a polyphosphate blend having a short average effective chain length. Methods of applying an oral care formulation comprising a linear polyphosphate or a polyphosphate blend having a short average effective chain length are also disclosed.

### FIELD OF THE INVENTION

The present invention relates to oral care formulations comprising blended short chain polyphosphates, or, alternatively, blends of polyphosphates having a short average effective chain length, which, in various embodiments, provide tooth whitening, stain protection, decreased tooth sensitivity, strengthened tooth enamel, and remineralization and that are effective across various delivery forms.

### BACKGROUND OF INVENTION

Oral care products such as toothpastes and mouth rinses are routinely used by consumers as part of their oral care hygiene regimens. It is well-known that oral care products can provide therapeutic, hygienic and cosmetic benefits to consumers. Therapeutic benefits may include caries prevention, gingivitis prevention, and sensitivity control. Hygienic benefits may include the control of plaque and breath freshening. Cosmetic benefits may include removal and prevention of tooth stain, tooth whitening, and overall improvement in mouth feel.

The addition of flavoring components, humectants, or other ingredients designed to increase aesthetic desirability of compositions for oral care products is common.

While previous oral care products have addressed some of the efficacy issues related to stain protection and tooth whitening, there continues to be a need for effective oral care products which can provide tooth whitening, stain removal, decreased tooth sensitivity, strengthened tooth enamel, and tooth remineralization. There also continues to be a need for alternatives to the peroxide-based formulas currently in use.

What is needed is an oral care formulation which can provide, in various embodiments, tooth whitening, superior stain protection following whitening, reduced tooth sensitivity, strengthened tooth enamel and provide tooth remineralization.

### SUMMARY OF THE INVENTION

Embodiments of the inventions disclosed herein are directed to oral care formulations containing short chain polyphosphates or polyphosphate blends having short effective chain length which are present in sufficient concentration to render the formulations effective for tooth whitening, stain protection, and other benefits. Surprisingly, it has been discovered that heretofore unutilized short-chain polyphosphates and short-effective-chain length polyphosphate blends provide such formulations with unexpected benefits for the users. In order to achieve an effective short-average chain length (number average chain length) used in the oral care formulations, unique polyphosphate blends have been developed which consist of either cyclic or linear polyphosphates or combinations of both. Such formulations provide for, in addition to tooth whitening, improved stain protection, decreased tooth sensitivity, strengthened tooth enamel, and tooth remineralization.

The oral care formulations, embodiments of which are disclosed herein, may be applied in the form of a gel, wrap, strip, applicator, rinse, toothpaste, tablet, or effervescent agent, for example and without limitation.

Oral care formulations as disclosed herein may comprise a polyphosphate blend having an average effective chain length of from about 3 to about 7.5 repeating units. The polyphosphate blend comprises a cyclic phosphate and a linear polyphosphate. In yet another embodiment, the formulation comprises a short chain polyphosphate which has an effective chain length of from about 3 to about 7.5 repeating units. The formulation of the embodiment further, optionally, comprises a humectant, a carbomer, an alkali metal hydroxide (buffering agent), xanthan gum (pseudoplastic agent), a flavorant, and, a sweetener. Such oral care formulations may further comprise an adhesive, a surfactant, or a lubricant. The pH of the oral care formulation is typically in the range of about 4 to about 6.5, however other pH values may be used when appropriate. Further embodiments of the formulations may comprise an amino acid or a calcium phosphate. Oral care formulations made according to the principles of the present invention may also comprise hydrogen peroxide.

The present invention further relates to methods for making an oral care formulation comprising a short chain polyphosphate or a polyphosphate blend having a short average effective chain length and methods of applying an oral care formulation comprising a short chain polyphosphate or a polyphosphate blend having a short average effective chain length. A method of preparing an oral care formulation as disclosed herein, wherein the oral care formulation comprises a polyphosphate blend, the polyphosphate blend comprises a cyclic and linear polyphosphate, and the polyphosphate blend has a short average chain length, comprises adding water to the polyphosphate blend in a mixing vessel along with a buffering agent to form a polyphosphate active ingredient mixture, mixing carbomer, humectant, and pseudoplastic agent to form a carbomer mixture, and adding the polyphosphate active ingredient mixture to the carbomer mixture. Additionally, a method of providing oral care active agents to an oral cavity comprises contacting a subject's dental enamel surfaces and mucosa in the mouth with an oral care formulation as disclosed herein, wherein the oral care formulation comprises a polyphosphate blend, the polyphosphate blend further comprises a cyclic and linear polyphosphate, and wherein the polyphosphate blend has a short average chain length.

### BRIEF DESCRIPTION OF THE DRAWINGS

FIG. 1 illustrates the results of the whitening performance evaluation for formulations of the present invention as a toothpaste booster after three rounds of treatment and compared to the performance of whitening toothpaste alone;

FIG. 2 illustrates the results of the whitening performance evaluation for formulations of the present invention as a toothpaste booster after seven rounds of treatment and compared to the performance of whitening toothpaste alone;

FIG. 3 illustrates the results of the whitening performance evaluation for formulations of the present invention in gel form as compared to hydrogen peroxide formulations after three rounds of treatment and compared to the performance of whitening toothpaste alone;

FIG. 4 illustrates the results of the whitening performance evaluation for formulations of the present invention in gel form as compared to hydrogen peroxide formulations after seven rounds of treatment and compared to the performance of whitening toothpaste alone;

FIG. 5 illustrates the change in whiteness performance for formulations of the present invention as compared to hydrogen peroxide formulations and compared to the performance of whitening toothpaste alone.

FIG. 6 illustrates the results of the whitening performance evaluation for formulations of the present invention in gel form as compared to hydrogen peroxide formulations after three rounds of treatment;

FIG. 7 illustrates the results of the whitening performance evaluation for formulations of the present invention in gel form as compared to hydrogen peroxide formulations after seven rounds of treatment; and

FIG. 8 illustrates the results of the whitening performance evaluation for formulations of the present invention in a strip or wrap format compared to performance of gel formulations of the present invention and to performance of a competing white strip product after three rounds of treatment; and

FIG. 9 illustrates the results of the whitening performance evaluation for formulations of the present invention in a strip or wrap format compared to performance of gel formulations of the present invention and to performance of a competing white strip product after seven rounds of treatment.

### DETAILED DESCRIPTION OF THE PREFERRED EMBODIMENTS

Embodiments of the inventions disclosed herein are directed to oral care formulations containing short chain polyphosphates or polyphosphate blends having short average effective chain lengths. Surprisingly, it has been discovered that heretofore unutilized short-chain polyphosphates and short effective chain length polyphosphate blends provide such formulations with unexpected benefits for the users. Formulations of the present invention which comprise a polyphosphate blend have an average effective chain length of from about 3 to about 7.5 repeating units. In order to achieve the average effective chain length (number average chain length) used in embodiments of the inventions disclosed herein, unique polyphosphate blends have been developed which consist of either cyclic or linear polyphosphates or combinations of both. Other formulations of the present invention comprise a polyphosphate having an effective chain length of from about 3 to about 7.5 repeating units. Efficacy testing, as provided herein below, shows that the oral care formulations disclosed herein provide for, in addition to tooth whitening, decreased tooth sensitivity, strengthened tooth enamel, and tooth remineralization.

The oral care formulations disclosed herein and further embodiments thereof, are products which in the ordinary course of usage, are not intentionally swallowed, but are rather retained in the oral cavity for a time sufficient to contact all of the dental surfaces and/or oral tissues and to provide for tooth whitening, decreased tooth sensitivity, strengthened tooth enamel, and/or tooth remineralization.

The oral care formulations disclosed herein and further embodiments thereof may be applied in the form of a gel, wrap, strip, applicator, rinse, toothpaste, tablet, or effervescent agent, for example and without limitation.

Oral care formulations of the present invention may comprise a short chain polyphosphate or a polyphosphate blend having a short average effective chain length of from about 3 to about 7.5 repeating units. The formulation further, optionally, comprises a carrier base acceptable for oral compositions. The carrier base acceptable for oral compositions optionally comprises a humectant, a carbomer, an alkali metal hydroxide (buffering agent), a pseudoplastic agent such as xanthan gum, a flavorant, and/or, a sweetener. A carrier base acceptable for oral compositions may contain any or all of the optional components identified above as will be known to a person of ordinary skill in the art. The pH of the formulation of the present invention is typically in the range of about 4 to about 6.5, however other pH values may be employed as appropriate to the applied use of the oral care composition. Further embodiments of the formulation may include an amino acid. Still further embodiments of the oral care formulation may include a calcium phosphate. Additional embodiments of the oral care formulation may include surfactants, adhesives, or lubricants as are known in the art. Oral care formulations made according to the principles of the present invention may also comprise hydrogen peroxide.

### Polyphosphate Blend

It has been discovered that a polyphosphate blend having a heretofore unutilized short average effective chain length is useful as a surface active agent for tooth whitening. Polyphosphates useful in embodiments of the oral care formulations disclosed herein may consist of multiple phosphate molecules arranged in a linear and/or cyclic configuration. In one embodiment, such polyphosphates are made by blending linear and cyclic polyphosphates with sufficient concentration to yield a specific average chain length of about 3 to about 7.5 repeating units. The polyphosphates, in a still further embodiment, may also be of branched form.

The ratios of cyclic polyphosphate to linear polyphosphate useful in the oral care formulations disclosed herein are from about 1:4 to about 3:2 expressed as mass of linear polyphosphate to mass of cyclic polyphosphate. It is preferred that such ratio be from about 1:2 to about 6:5. It is still further preferred that such ratio be from about 45:55 to about 55:45.

Polyphosphate compounds useful in the oral care formulations of the present invention may include pyrophosphate, trimetaphosphate, tripolyphosphate, and ultrametaphosphate for example. In various embodiments of the invention, the polyphosphate active ingredient may be a linear polyphosphate compound such as sodium tripolyphosphate or a blended polyphosphate such as ultrametaphosphate and a linear polyphosphate or ultrametaphosphate and a cyclic polyphosphate. Surprisingly, the efficacy of oral care formulations are improved and enhanced as a result of the use of the novel short average effective chain length polyphosphate blends disclosed herein. Short average effective chain length may also be referred to herein as short average chain length. Embodiments of polyphosphate blends in the formulations described herein may comprise predominantly cyclic combinations of polyphosphate compounds, or “cyclic/cyclic combination polyphosphate blends”, and predominantly linear combinations of polyphosphate compounds, or “cyclic/linear combination polyphosphate blends,” for example and without limitation.

As one example, (Example A) a predominantly cyclic combination, or a “cyclic/cyclic combination polyphosphate blend,” comprises sodium tripolyphosphate (1.3% w/w), sodium pyrophosphate (0.3% w/w), sodium trimetaphosphate (48.5% w/w) and ultrametaphosphate (50% w/w). The cyclic/cyclic combination polyphosphate blend described here is formulated to have a short average effective chain length of about 5.65. In an alternate example (Example B), a predominantly linear combination, or a “cyclic/linear combination polyphosphate blend” may be made according to the principles disclosed herein whereby sodium tripolyphosphate is substituted for the sodium trimetaphosphate in the polyphosphate blend of Example A at a sufficient level in combination with ultrametaphosphate to achieve an short average effective chain length of about 6.55. As will be readily apparent to those of ordinary skill in the art, other components of the polyphosphate blend may be adjusted to vary the chain length of the combination polyphosphate blend. Additionally, other embodiments of cyclic/cyclic combination polyphosphate blends and cyclic/linear combination polyphosphate blends will be evident to those of ordinary skill in the art. For the examples further provided herein below, we refer to two polyphosphate blends which are used in the example formulations. One is the cyclic/cyclic combination polyphosphate blend of Example A having a chain length of about 5.65, and the second is the cyclic/linear combination polyphosphate blend of Example B having a chain length of about 6.55 Also, in the examples below we refer to a linear polyphosphate having an effective chain length of about 4.0.

In making the polyphosphates blends in accordance with the principles disclosed herein, the controlling factor is the average effective chain length being kept to a range of values from about 3.0 to about 7.5 repeating units. Thus, blends of polyphosphates which are predominantly cyclic combinations, or cyclic/cyclic combination polyphosphate blends, and predominantly linear combinations, or cyclic/linear combination polyphosphate blends, are blended so that the average effective chain length is kept to a range of from about 3.0 to about 7.5 repeating units.

The effective average chain length for the polyphosphates used in embodiments of the oral care formulations disclosed herein were determined by ion chromatography. Reference is made to DIONEX “Application Update 172,” April 2010, which provides a detailed description of the method used herein to determine effective average chain length of phosphates.

The following parameters and guidelines were used:

Column: IONPAC AS16 analytical, 2×250 mm;
coupled with an IONPAC AG16 guard, 2×50 mm

Eluent: EGC II KOH, so mM from 0 to 2 minutes and
50 to 100 mM from 2 to 80 minutes (gradient curve 3

Run time: 120 minutes

Temperature: 30° C.

Flow Rate: 0.25 mL/minute

Sample Volume: 10 μL

Detection: Suppressed conductivity, ASRS 300
external water mode, CRD 200

Samples were prepared by diluting 1:100 with deionized water and then filtered prior to injection. Standards were prepared from 1000 mg/L stock standard solutions by dissolving the appropriate weight (Orthophosphate—0.809 g, Pyrophosphate—0.268 g, Trimetaphosphate—0.129 g, Triphosphate—0.145 g) into separate 100 mL volumetric flasks in deionized water to form the corresponding “Intermediate.” Separately, 30 mL of Ortho and Pyro stock standards were diluted to 100 mL with deionized water. 5 mL Ortho Intermediate, 10 mL Pyro Intermediate, 10 mL Trimeta Stock, and 10 mL Triphosphate Stock were combined into a 100 mL volumetric flask and diluted to volume with deionized water.

For each of the standards prepared according to the description above, the elution time for each type of phosphate was determined using the ion chromatography method described above. Samples of polyphosphate blends made according to the principles disclosed herein were then prepared and subjected to the ion chromatography analysis described above. The effective average chain length was then calculated for the specific polyphosphate blends used in the oral care formulations. The effective average chain length was calculated based on the percent area under the detection curve each identified component of a blend occupied as compared to the total area under the elution curve.

The following components and corresponding effective chain lengths were used in the calculations.

TABLE 1

| Component | Effective Chain Length |
|---|---|
| orthophosphate | 1 |
| pyrophosphate | 2 |
| trimetaphosphate | 3 |
| tetrametaphosphate | 3 |
| tripolyphosphate | 4 |
| tetrapolyphosphate | 4 |
| hexametaphosphate | 6 |

For purposes of illustrating the ion chromatography method employed for determining effective average chain length, the following example is provided. A sample of a blended polyphosphate having a cyclic/cyclic combination (Example A above) was prepared according to the procedure outlined above and injected into an ion chromatograph set up according to the parameters also outlined above. The area under the detection curve was determined for each of the species identified in Table 1. The percent each individual area occupied under the whole of the detection curve was calculated. Based on the effective chain length for each phosphate species detected an average chain length for the sample was calculated. The pertinent results of the ion chromatography analysis are summarized in Table 2 below.

TABLE 2

| | Blended | | |
|---|---|---|---|
| | Cyclic/Cyclic | | |
| | Combination | | |
| | (Example A) | | |
| Phosphate | nᵢ | areaᵢ | (nᵢ*areaᵢ) |
| Orthophosphate | 1 | 2.564 | 2.564 |
| Pyrophosphate | 2 | 4.284 | 8.568 |
| Trimetaphosphate | 3 | 71.808 | 215.424 |
| Tripolyphosphate | 3 | 2.865 | 8.595 |
| Tetrametaphosphate | 4 | 0.903 | 3.612 |
| Tetrapolyphosphate | 4 | 3.278 | 13.112 |
| Hexametaphosphate | 6 | 0.423 | 2.538 |
| | 5 | 3.322 | 16.61 |
| | 6 | 3.038 | 18.228 |
| | 7 | 2.966 | 20.762 |
| | 8 | 2.835 | 22.68 |
| | 9 | 2.805 | 25.245 |
| | 10 | 2.617 | 26.17 |
| | 11 | 2.479 | 27.269 |
| | 12 | 2.302 | 27.624 |
| | 13 | 2.156 | 28.028 |
| | 14 | 2.039 | 28.546 |
| | 15 | 1.882 | 28.23 |
| | 16 | 1.744 | 27.904 |
| | 17 | 1.597 | 27.149 |
| | 18 | 1.47 | 26.46 |
| | 19 | 1.34 | 25.46 |
| | 20 | 1.226 | 24.52 |
| | 21 | 1.116 | 23.436 |
| | 22 | 1.018 | 22.396 |
| Σ | | 124.077 | 701.13 |

where, nᵢ = chain length
areaᵢ = area under the detection peak for a given phosphate
Σ = sum of areaᵢ or nᵢ*areaᵢ, respectively.

The effective average chain length is calculated as:

effective average chain length=Σnᵢ*areaᵢ/Σareaᵢ.

The effective average chain length for the example in Table 2 is:

effective average chain length=701.13/124.077=5.65.

Accordingly, for the cyclic/cyclic combination polyphosphate blend sample analyzed, an effective average chain length of 5.65 was determined.

Polyphosphate chain length for oral care formulations made according to the principles disclosed herein may also be determined using phosphorous-31 (³¹P) NMR. ³¹P NMR is considered the standard test for determining polyphosphate chain length. Common methods for determining number-average chain length (n) are known. For example, a ³¹P NMR standard method is reported in: J. C. MacDonald and M. Mazurek, J. Magn. Reson., 72 (1987) 48.

The procedure for the NMR measurements was a modification of the one described in the Journal of Chromatography A, 688 (1994) 89-95. In the ³¹P NMR analyses performed on oral care formulations made according to the principles disclosed herein, samples were placed a 5 mm NMR tubes and analyzed in a 400 MHz Bruker Avance III instrument furnished with a BBO probe. The following parameters were used.
Pulse: 15 degrees;
Repetition rate: 7 sec (0.8 sec acquisition time plus 6.2 sec delay); and
Transients: 900.

Samples were prepared for ³¹P NMR analysis by adding 4 mL water to a 55±5 mg sample of the polyphosphate blend to be tested. The thus prepared solution was then adjusted to pH of 9.5±0.5 by the addition of 0.1 M NaOH (ca. 0.7 mL). To 0.5 mL of said solution was added 0.1 mL of deuterium oxide for locking purposes. The thus prepared solution was measured immediately after it was prepared. The polyphosphates tested via NMR analysis include a sodium tripolyphosphate, a cyclic/cyclic combination polyphosphate blend, and a cyclic/linear combination polyphosphate blend.

As an example of the measurements obtained, the spectra provided the following results:

TABLE 3

| Sample | I | E | O | ñ(NMR) |
|---|---|---|---|---|
| 1 | 30.03 | 21.68 | 1.00 | 4.45 |

Thus, Sample 1 was found to have an effective average chain length of 4.45. As set forth in Table 3, O=ortho resonance, E=end group resonance, and I=internal resonance for the spectra.

### Humectant

Another component of an embodiment of the oral care formulations may be a humectant. The humectant keeps the formulation from hardening upon exposure to air and certain humectants may impart desirable sweetness of flavor to formulations as well. Suitable humectants for use in the formulation include, for example and without limitation, glycerin, sorbitol, polyethylene glycol, and xylitol. The humectant may comprise from about 0 to about 70%, and in one embodiment, from about 24% to about 45%, by weight of the formulation.

### Carbomer

The present oral care formulations may further include a carbomer. The carbomer as used herein refers to an agent which can be used to thicken the formulation at a low concentration and to produce a wide range of viscosities and flow properties in creams, gels, and oral suspensions, for example. Carbomers further provide textural benefit. In the formulation described herein, the carbomer may comprise from about 2 to about 5%, by weight of the formulation.

### Buffering Agent

The present oral care formulations may contain a buffering agent. Buffering agents, as used, herein, refer to agents that can be used to adjust the pH of the compositions to a range of about pH 3 to about pH 10. The formulation here will typically have a slurry pH here of from about 4 to about 10, preferably from about 4.0 to about 8, and more preferably from about 4.0 to about 6.5. The buffering agents may include alkali metal hydroxides, carbonates, sesquicarbonates, borates, silicates, phosphates, imidazole, and mixtures thereof. Specific buffering agents include sodium hydroxide, potassium hydroxide, alkali metal carbonate salts, sodium carbonate, imidazole, citric acid, and sodium citrate, for example and without limitation. Buffering agents are used at a level of from about 0.1% to about 30%%, preferably about 1% to about 10% and more preferably about 5 to about 9%, by weight of the present formulation.

### Pseudoplastic Agent

Oral care formulations of the present invention may further contain a pseudoplastic material or other thickening agent to provide a desired consistency and allow for a wide range of delivery forms. Suitable pseudoplastic materials may include without limitation carboxyvinyl polymers, carrageenan, hydroxyethyl cellulose, and water-soluble salts of cellulose ethers. Natural gums such as xanthan gum, gum arabic, and gum karaya, for example and without limitation, may also be used. In the present formulation, xanthan gum is used as a pseudoplastic agent. Thickening agents can be used in an amount from about 0.01% to about 15%, preferably about 0.5% to about 1.0%, by weight of the formulation.

### Flavorant and Sweetener

A flavorant can also be added to the oral care formulations. Suitable flavoring components may include oil of wintergreen, oil of peppermint, oil of spearmint, menthol, cinnamon, vanillin, lemon, orange, and the like. Coolants may also be part of the flavorant. In the formulation of the present invention, suitable coolants may include oil of peppermint. The flavorant is generally used in the present invention at levels of up to about 4% and preferably in one embodiment from about 0.5% to about 1.0%, by weight of the formulation. Sweeteners may also be added to the formulations. Sweeteners may include neotame, saccharin, dextrose, sucrose, lactose, maltose, levulose, aspartame, sodium cyclamate, D-tryptophan, dihydrochalcones, acesulfame, and mixtures thereof. A sweetener is generally added to an embodiment of the formulation at levels of from about 0.005% to about 5%, by weight of the composition. In another embodiment, the sweetener is added at a level of from about 0.05% to about 0.1%, by weight of the composition.

Water employed in the preparation of commercially suitable oral care compositions should preferably be of low ion content and free of organic impurities. Water will generally comprise from about 5% to about 70%%, and in one embodiment from about 35% to about 61%% by weight of the formulation. The amounts of water include the free water which is added plus that which is introduced with other materials such as with sorbitol, surfactant solutions, and/or color solutions.

### Amino Acid

Alternative embodiments of the oral care formulations may further include an amino acid. An amino acid, in zwitterionic form, may stabilize the chelating process driven by the polyphosphate ingredient to provide the whitening effect to the surface of the teeth. Suitable amino acids may include glycine, lysine, arginine, and proline for example. In an embodiment, proline may be added to the formulation disclosed herein. An amino acid may be present up to a level of about 5% by weight of the formulation, and in one embodiment from about 0.5% to about 2% by weight of the formulation.

### Method of Preparation

As described herein, the polyphosphate active ingredient for the present invention may comprise a linear polyphosphate, a cyclic/cyclic combination polyphosphate blend, a cyclic/linear combination polyphosphate blend or other polyphosphate blends such that the polyphosphate or polyphosphate blend has an average effective chain length of from about 3 to about 7.5 repeating units. Water is added to the polyphosphate active ingredient in a mixing vessel along with a buffering agent to form a polyphosphate active ingredient mixture. The carbomer, humectant, and pseudoplastic agent are mixed in a separate step to form a carbomer mixture. The polyphosphate active ingredient mixture is subsequently added to the carbomer mixture. The final solution is mixed together to allow for dissolution. In an alternative embodiment, the carbomer mixture can also be added to the wetted, dissolved polyphosphate active ingredient via a slow process to minimize clumping and air bubbles in the mixture. This process includes use of a disperser and a mixing vessel designed for mixing thick, high viscosity gels.

### Method of Treatment

The formulations of the present invention may be in the form of toothpastes, topical oral gels, mouth rinses, denture products, mouth sprays, lozenges, oral tablets, floss or the like.

The present invention also relates to methods for improved tooth whitening, reduced tooth sensitivity, strengthened enamel, and tooth remineralization. The benefits of the formulations may increase over time when the composition is repeatedly used.

The method of treatment herein comprises contacting a subject's dental enamel surfaces and mucosa in the mouth with the oral care formulations made according to the principles disclosed herein. The method of treatment may be by brushing with a dentrifice or rinsing with dentrice slurry or mouth rinse. Other methods include contacting a topical oral gel, dentures product, mouth spray, or other form containing an oral care formulation with the subject's teeth and oral mucosa. The subject may be any person or lower animal whose tooth surface contacts the oral care formulation made according to the principles disclosed herein. It should be understood that the present invention relates not only to methods for delivering the formulation to the oral cavity of a human, but also to methods of delivering these compositions to the oral cavity of other animals, e.g., household pets or other domestic animals, or animals kept in captivity.

### EXAMPLES

The following non-limiting examples further illustrate and describe the embodiments of the oral care formulations described herein. The examples are given solely for the purpose of illustration and are not to be construed as limiting the scope of the present invention, as many variations thereof are possible without departing from the spirit and scope of the invention.

Tables 4-12 below set forth sample oral care formulations based on the principles disclosed herein. The % w/w values provided below are the weight percent of each component based on of the weight of the total formulation.

TABLE 4
Example 1-Linear Polyphosphate Formulation

| Ingredient | % w/w | Purpose |
|---|---|---|
| Water | 46 | |
| Glycerin | 36.3 | Humectant |
| Sodium Tripolyphosphate | 6.5 | Whitening |
| (Chain length about 4) | | |
| Carbomer | 4.5 | Rheology |
| | | modifier |
| Sodium Hydroxide | 5.4 | Buffering |
| Xanthan Gum | 0.6 | Pseudoplastic |
| | | Agent |
| Peppermint Flavor | 0.6 | Flavoring |
| Neotame | 0.1 | Sweetener |
| | 100 | |

TABLE 5
Example 2- Cyclic/Cyclic Combination Polyphosphate Formulation

| Ingredient | % w/w | Purpose |
|---|---|---|
| Water | 44.1 | |
| Glycerin | 35.6 | Humectant |
| Sodium Hydroxide | 8.6 | Buffering |
| Cyclic/Cyclic Combination | 6 | Whitening/Stain |
| Polyphosphate Blend Chain | | Protection |
| Length about 5.65 (Example A) | | |
| Carbomer | 4.4 | Rheology |
| | | modifier |
| Xanthan Gum | 0.6 | Pseudoplastic |
| | | Agent |
| Peppermint Flavor | 0.6 | Flavoring |
| Neotame | 0.1 | Sweetener |
| | 100 | |

TABLE 6
Example 3- Cyclic/Linear Combination Polyphosphate Formulation

| Ingredient | % w/w | Purpose |
|---|---|---|
| Water | 46.6 | |
| Glycerin | 35.8 | Humectant |
| Cyclic/Linear Combination | 6.6 | Whitening/Stain |
| Polyphosphate Blend (Chain | | Protection |
| Length about 6.55) (Example B) | | |
| Sodium Hydroxide | 5.3 | Buffering |
| Carbomer | 4.4 | Rheology |
| | | modifier |
| Xanthan Gum | 0.6 | Pseudoplastic |
| | | Agent |
| Peppermint Flavor | 0.6 | Flavoring |
| Neotame | 0.1 | Sweetener |
| | 100 | |

TABLE 7
Example 4- Linear polyphosphate Formulation

| Ingredient | % w/w | Purpose |
|---|---|---|
| Water | 45.0 | |
| Glycerin | 36.3 | Humectant |
| Sodium Tripolyphosphate | 6.5 | Whitening |
| (Chain length about 4) | | |
| Carbomer | 4.5 | Rheology |
| | | modifier |
| Sodium Hydroxide | 5.4 | Buffering |
| Proline | 1.0 | Therapeutic |
| | | agent |
| Xanthan Gum | 0.6 | Pseudoplastic |
| | | Agent |
| Peppermint Flavor | 0.6 | Flavoring |
| Neotame | 0.1 | Sweetener |
| | 100.0 | |

TABLE 8
Example 5- Cyclic/Cyclic Combination Polyphosphate Formulation

| Ingredient | % w/w | Purpose |
|---|---|---|
| Water | 42.9 | |
| Glycerin | 35.8 | Humectant |
| Sodium Hydroxide | 8.6 | Buffering |
| Cyclic/Cyclic Combination | 6.0 | Whitening/Stain |
| Polyphosphate Blend Chain | | Protection |
| Length about 5.65 (Example A) | | |
| Carbomer | 4.4 | Rheology |
| | | modifier |
| Proline | 1.0 | Therapeutic |
| | | agent |
| Xanthan Gum | 0.6 | Pseudoplastic |
| | | Agent |
| Peppermint Flavor | 0.6 | Flavoring |
| Neotame | 0.1 | Sweetener |
| | 100.0 | |

TABLE 9
Example 6- Cyclic/Linear Combination Polyphosphate Formulation

| Ingredient | % w/w | Purpose |
|---|---|---|
| Water | 44.8 | |
| Glycerin | 36.3 | Humectant |
| Cyclic/Linear Combination | 6.6 | Whitening/Stain |
| Polyphosphate Blend (Chain | | Protection |
| Length about 6.55) (Example B) | | |
| Sodium Hydroxide | 5.5 | Buffering |
| Carbomer | 4.5 | Rheology |
| | | modifier |
| Proline | 1.0 | Therapeutic |
| | | agent |
| Xanthan Gum | 0.6 | Pseudoplastic |
| | | Agent |
| Peppermint Flavor | 0.6 | Flavoring |
| Neotame | 0.1 | Sweetener |
| | 100.0 | |

TABLE 10
Example 7- Linear polyphosphate Formulation

| Ingredient | % w/w | Purpose |
|---|---|---|
| Water | 44.9 | |
| Glycerin | 36.2 | Humectant |
| Sodium Tripolyphosphate | 6.5 | Whitening |
| (Chain length about 4) | | |
| Carbomer | 4.5 | Rheology |
| | | modifier |
| Sodium Hydroxide | 5.4 | Buffering |
| Proline | 1.0 | Remineralization |
| | | aid/Therapeutic |
| | | agent |
| Xanthan Gum | 0.6 | Pseudoplastic |
| | | Agent |
| Peppermint Flavor | 0.6 | Flavoring |
| Calcium Pyrophosphate | 0.2 | Remineralization |
| Neotame | 0.1 | Sweetener |
| | 100.0 | |

TABLE 11
Example 8 - Cyclic/Cyclic Combination
Polyphosphate Formulation

| Ingredient | % w/w | Purpose |
|---|---|---|
| Water | 45.4 | |
| Glycerin | 36.2 | Humectant |
| Sodium Hydroxide | 5.4 | Buffering |
| Cyclic/Cyclic Combination | 6.0 | Whitening/Stain |
| Polyphosphate Blend Chain | | Protection |
| Length about 5.65 (Example A) | | |
| Carbomer | 4.5 | Rheology |
| | | modifier |
| Proline | 1.0 | Remineralization |
| | | aid/Therapeutic |
| | | agent |
| Xanthan Gum | 0.6 | Pseudoplastic |
| | | Agent |
| Peppermint Flavor | 0.6 | Flavoring |
| Calcium Pyrophosphate | 0.2 | Remineralization |
| Neotame | 0.1 | Sweetener |
| | 100.0 | |

TABLE 12
Example 9- Cyclic/Linear Combination Polyphosphate Formulation

| Ingredient | % w/w | Purpose |
|---|---|---|
| Water | 45.0 | |
| Glycerin | 36.2 | Humectant |
| Cyclic/Linear Combination | 6.6 | Whitening/Stain |
| Polyphosphate Blend (Chain | | Protection |
| Length about 6.55) (Example B) | | |
| Sodium Hydroxide | 5.3 | Buffering |
| Carbomer | 4.4 | Rheology |
| | | modifier |
| Proline | 1.0 | Remineralization |
| | | aid/Therapeutic |
| | | agent |
| Xanthan Gum | 0.6 | Pseudoplastic |
| | | Agent |
| Peppermint Flavor | 0.6 | Flavoring |
| Calcium Pyrophosphate | 0.2 | Remineralization |
| Neotame | 0.1 | Sweetener |
| | 100.0 | |

### Evaluation of Whitening Performance for Pearl Gel Formulation When Used as Toothpaste Booster

The in-vitro whitening performance of oral care formulations of Example 3 above (Ex. 3) and Example 2 above (Ex. 2) for use with non-whitening toothpaste compared with a control (carbomer placebo with no active polyphosphate ingredient) and whitening toothpaste was evaluated. Example 3 as described below and set forth in the Examples above comprises a cyclic/linear polyphosphate blend formulation having a chain length of about 6.55. Example 2 as described below and set forth in the Examples above comprises a cyclic/cyclic polyphosphate blend formulation having a chain length of about 5.65.

The study was set up with the following materials and methods:

Bovine teeth were etched up to the root in 100 ml of 1M HCl for 1 minute and then submerged (up to the root) in stain mixture (99% soy sauce, 0.5% ground coffee, 0.5% ground tea) for 24 hrs. at 40° C. at 70% RH. The teeth were removed from stain solution, grouped, and treated according to the application and regimen listed below, with 3 teeth tested per group:
Group 1A-1C: Placebo; 0.150 g+/−0.010 g
   30 min. each treatment, 7 treatments
Group 5A-5C: Example 2 (as toothpaste booster gel)+Non-whitening toothpaste; 0.330 g+/−0.020 g
   1 min. total brushing treatment (both products at once) to simulate 2× daily brushing for maximum of 30 seconds on any one tooth, 7 treatments
Group 6A-6C: Example 3 (as toothpaste booster gel)+Non-whitening toothpaste; 0.330 g+/−0.020 g
   1 min. total brushing (both products at once) to simulate 2× daily brushing for maximum of 30 seconds on any one tooth, 7 treatments
Group 7A-7C: Total Whitening toothpaste; 0.330 g+/−0.020 g
   1 min. total brushing to simulate 2× daily brushing for maximum of 30 seconds on any one tooth, 7 treatments

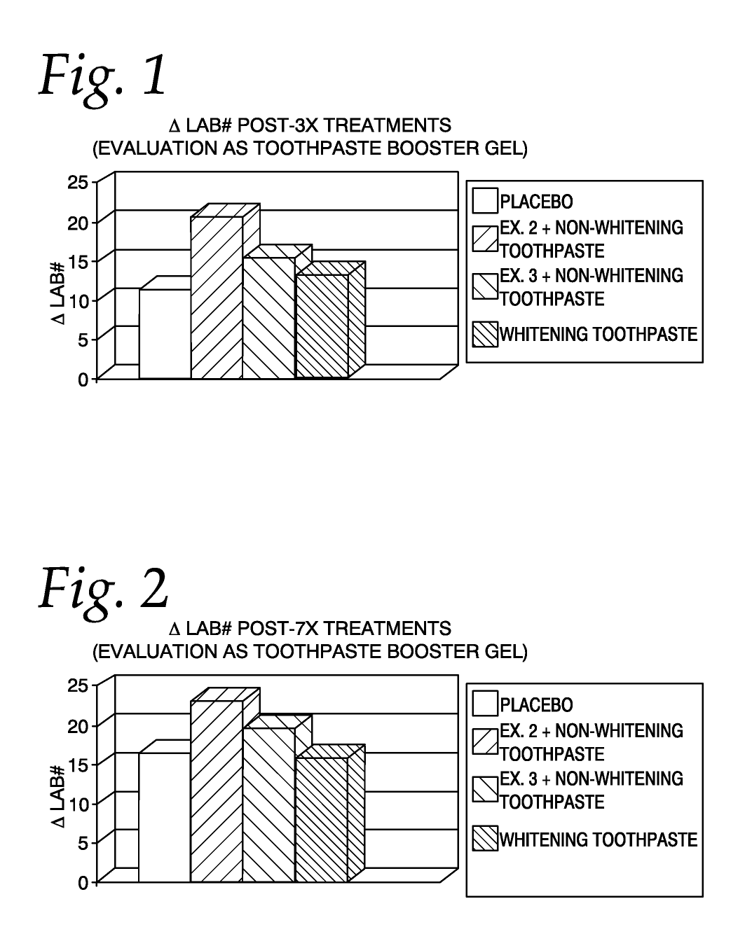

Images of each tooth were taken post-etching, post-staining, and post-each treatment using the X-RITE SHADEVISION System. FIG. 1 represents the whitening performance of formulas of Ex. 2 and Ex. 3 as a toothpaste booster post-3× treatments against the performance of whitening toothpaste alone while FIG. 2 represents post-7× treatments.

It is believed that the polyphosphate active(s) are effectively whitening via chelation. The amino acids in embodiments of the formulation of the present invention are believed to stabilize the chelating process because of the presence of the amino acid in zwitterionic form. Lysine, arginine and proline were evaluated in embodiments of the formulation of the present invention. In an embodiment, proline was selected for use because it is a component of enamel, and may enhance the remineralization process since it aids in the formation of high proline peptides (HPPs).

The mean % improvement post-7× treatments relating to use of formulas of Ex. 3 and Ex. 2 as a toothpaste booster gel vs. use of whitening toothpaste alone is 23% and 45%, respectively as compared to the use of whitening toothpaste alone.

The in-vitro whitening performance of formulas in Ex. 2 and Ex. 3 when used as toothpaste booster gels to non-whitening toothpaste was assessed in terms of change in LAB # or laboratory shades. This preliminary data indicates that using both formulas of Ex. 2 and Ex. 3 as a toothpaste booster gel with toothpaste improves tooth whitening vs. just brushing with whitening toothpaste alone.

### Evaluation of Whitening Performance for Gel Formulations Compared to Hydrogen Peroxide Formulations

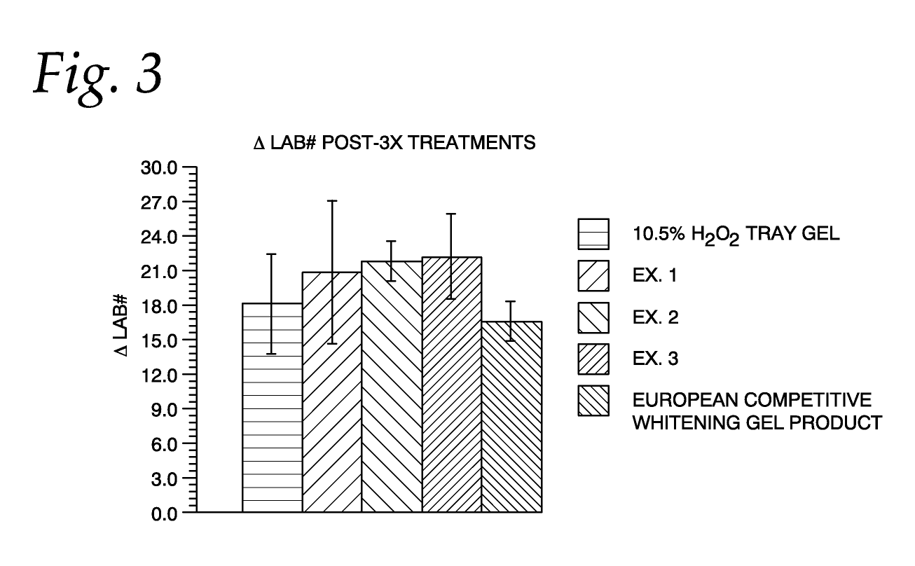

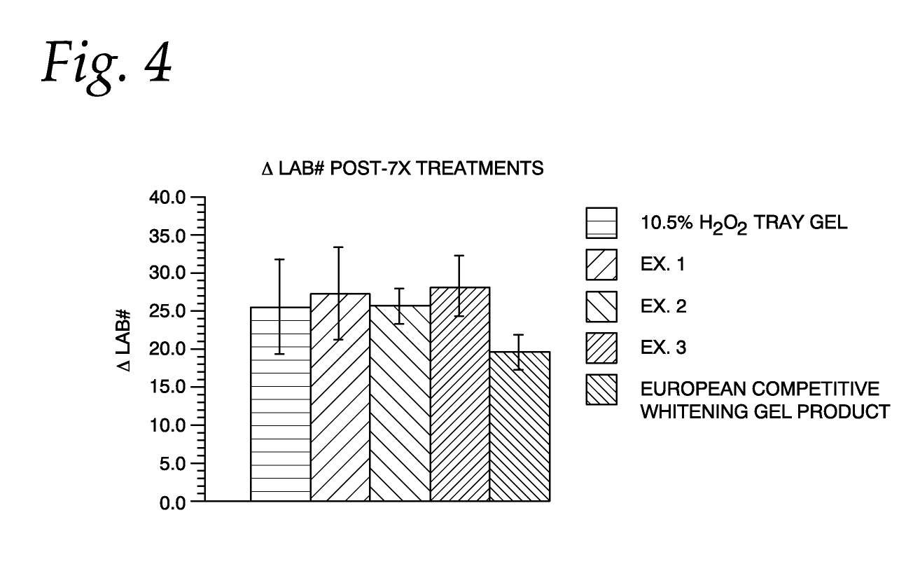

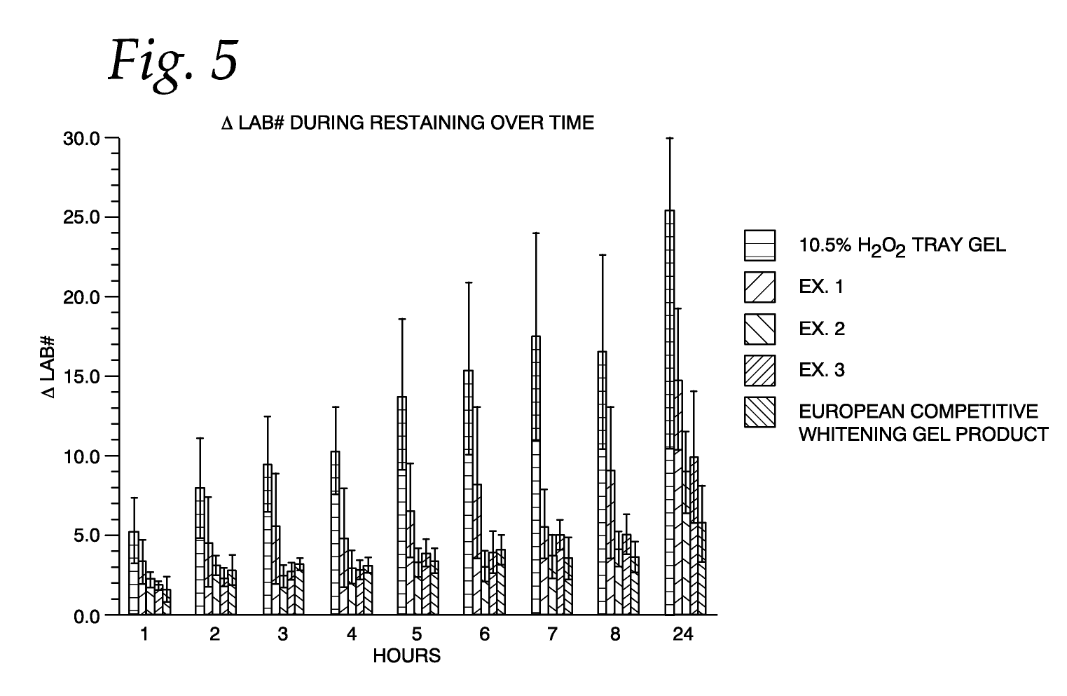

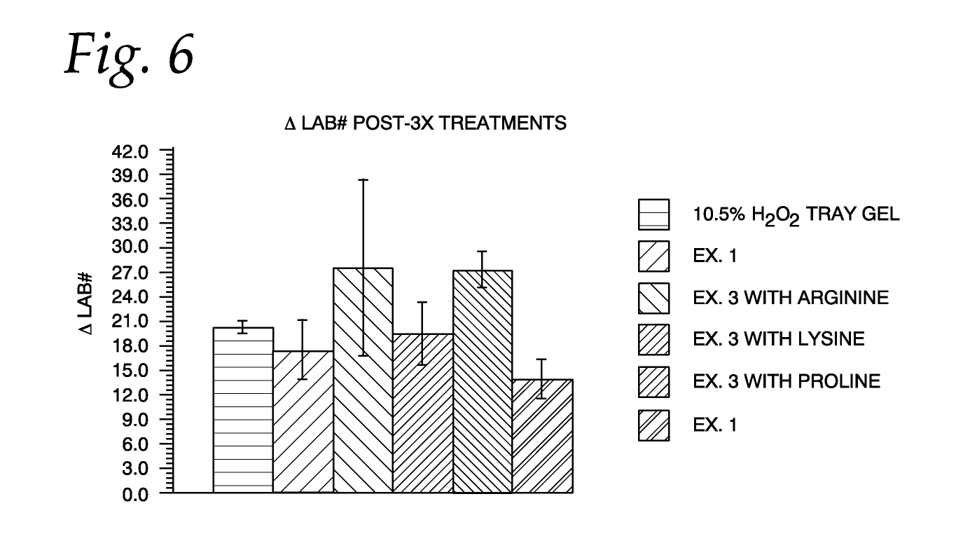

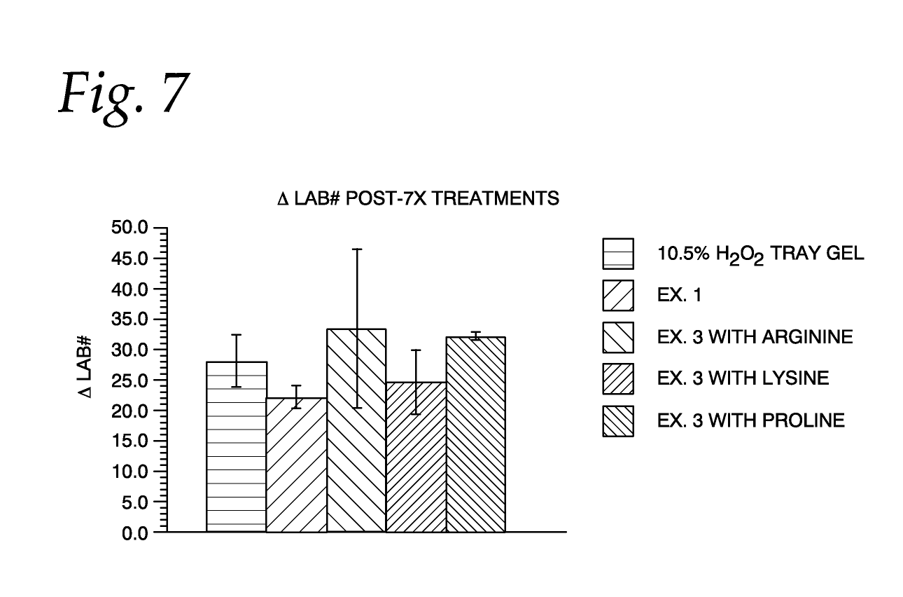

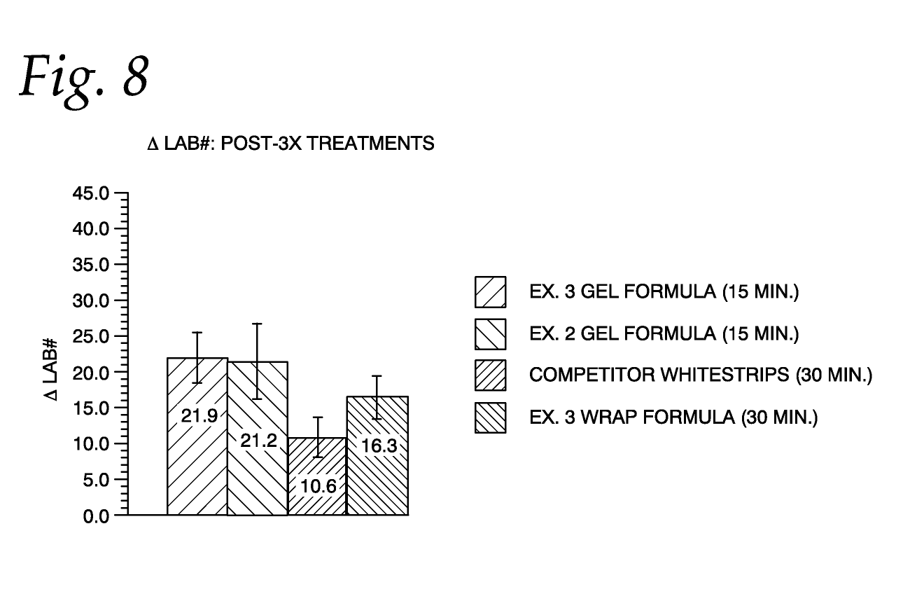

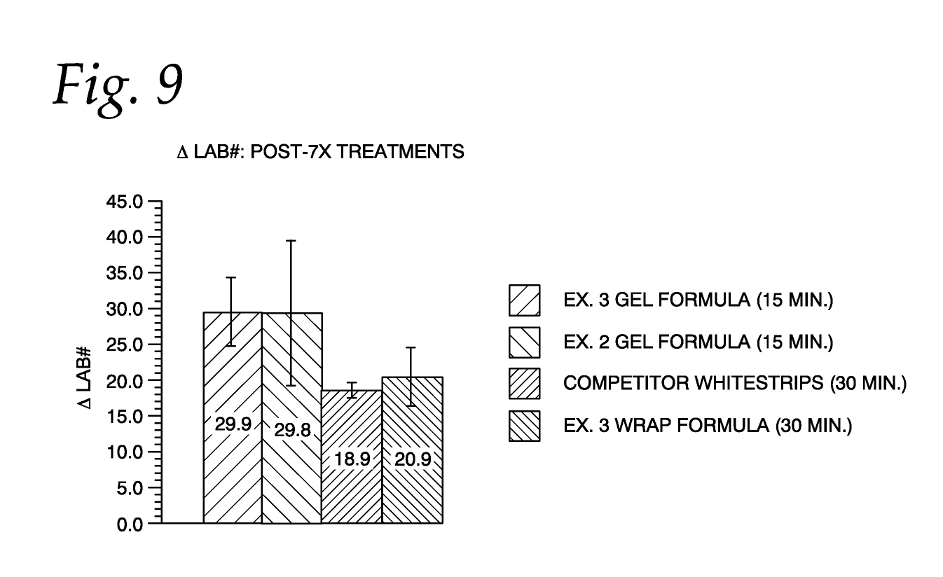

The in-vitro whitening performance of formulations of the present invention compared with hydrogen peroxide based gel formulations was evaluated. The methodology for the study was identical to that set forth above for the evaluation of the formulations as a toothpaste booster gel. FIGS. 3 and 4 represent the whitening performance of a linear polyphosphate formulation (Example 1 above, Ex. 1), a cyclic/cyclic polyphosphate blend formulation (Example 2, Ex. 2) and a cyclic/linear polyphosphate blend formulation (Example 3, Ex. 3) as compared to both whitening toothpaste alone and a hydrogen peroxide based formulation. FIG. 5 represents the change in whitening performance for a linear polyphosphate formulation (Ex. 1), a cyclic/cyclic polyphosphate blend formulation (Ex. 2) and a cyclic/linear polyphosphate blend formulation (Ex. 3) as compared to both a whitening toothpaste alone and a hydrogen peroxide based formulation. FIGS. 6 and 7 represent the whitening performance of linear polyphosphate formulations (Ex. 1) and cyclic/linear polyphosphate blend formulations (Ex. 3) with amino acid as compared to hydrogen peroxide based formulations after three and seven rounds of treatment respectively. Additionally, FIGS. 8 and 9 represent the whitening performance of a cyclic/cyclic polyphosphate blend formulation (Ex. 2) in a strip or wrap format compared with a gel cyclic/cyclic polyphosphate blend formulation (Ex. 2), a gel cyclic/linear polyphosphate blend formulation (Ex. 3), and a competing white strip product after three and seven rounds of treatment respectively.

### Formulations for Microbial Study and Summary of the Study Results

Embodiments of the oral care formulations were tested for antimicrobial effectiveness against five microorganisms. The formulations set forth above in Tables 4-12 were modified for the antimicrobial study with the addition of benzyl alcohol. Sample oral care formulations for the microbial study are set forth below in Tables 13-14. Sample oral care formulations modified for the microbial study include a cyclic/linear polyphosphate blend combination (Example B) and cyclic/cyclic polyphosphate blend combination (Example A). The results of the antimicrobial study are further laid out in Tables 15-17 below.

TABLE 13
Example #10 -Formulation modified for Microbial Study

| Ingredient | % w/w |
|---|---|
| cold water | 45.9 |
| 6N NaOH | 3.2 |
| Cyclic/cyclic | 6.0 |
| polyphosphate blend | |
| (Chain Length 5.65) (Example A) | |
| Neotame | <0.1 |
| Glycerin | 34.9 |
| Benzyl Alcohol | 0.8 |
| ETD2020 | 3.6 |
| Peppermint Flavor | 0.6 |
| Xanthan Gum | 0.6 |
| 6N NaOH | 4.4 |
| Total | 100.00 |

TABLE 14
Example #11 -Formulation modified for Microbial Study

| Ingredient | % w/w |
|---|---|
| cold water | 48.4 |
| 6N NaOH | 3.2 |
| Cyclic/linear | 6.6 |
| polyphosphate blend | |
| (Chain Length 6.55) (Example B) | |
| Neotame | <0.1 |
| Glycerin | 35.0 |
| Benzyl Alcohol | 0.8 |
| ETD2020 (Carbomer) | 3.6 |
| Peppermint Flavor | 0.6 |
| Xanthan Gum | 0.6 |
| 6N NaOH | 4.4 |
| Total | 100.00 |

The formulations of the present invention were tested for antimicrobial effectiveness against five microorganisms: Aspergillus niger, Candida albicans, Escherichia coli, Pseudomonas aeruginosa, and Staphylococcus aureus. The formulations under test were examined at inoculation, at 14 days after inoculation, and at 28 days after inoculation. The microbiological analyses were performed based on official methods referencing United States Pharmacopoeia 51, AOAC, and Microbiologics. Staphylococcus aureus results were not available after 28 days.

For each of the formulations under test, the growth of the five organisms examined in each formulation was identified at a level that was less than the control or at a level effectively indicating no growth. The results are provided in Tables 15-17 below.

TABLE 15
MICROORGANISM EVALUATED

| | | | | | | Total |
|---|---|---|---|---|---|---|
| | | | | | | Aerobic |
| | Aspergillus | Candida | Escherichia | Pseudomonas | Staphylococcus | Plate |
| FORMULATION | niger | albicans | coli | aeruginosa | Aureus | Controld |
| TESTED | (cfu/mL) | (cfu/mL) | (cfu/mL) | (cfu/mL) | (cfu/mL) | (cfu/mL) |
| Initial Test | Recovery: | Recovery: | Recovery: | Recovery: | Recovery: | Recovery: |
| Results (@ 1:1) | | | | | | |
| (Inoculated: | | | | | | |
| 6/8/11, 1 mL) | | | | | | |
| Gel Formulation = | NA | NA | <1 (No | <1 (No | NA | <1 (No |
| (Example 10) | | | growth) | growth) | | growth) |
| Gel Formulation = | NA | NA | <1 (No | <1 (No | NA | <1 (No |
| (Example 11) | | | growth) | growth) | | growth) |
| Standard = | NA | NA | 21/mL | 205/mL | NA | |
| Organism Control | | | | | | |

TABLE 16
MICROORGANISM EVALUATED

| | FORMULATION TESTED | | | | |
|---|---|---|---|---|---|
| | Aspergillus niger | Candida albicans | Escherichia coli | Pseudomonas aeruginosa | Staphylococcus Aureus |
| | (cfu/mL) | (cfu/mL) | (cfu/mL) | (cfu/mL) | (cfu/mL) |
| Initial Test Results | Recovery: | Recovery: | Recovery: | Recovery: | Recovery: |
| (@ 1:13) | | | | | |
| (Inoculated: Jun. 14, 2011, | | | | | |
| 1 mL) | | | | | |
| Gel Formulation | 135 | 87 | <3 (No | <3 (No | <3 (No |
| (Example 10) = | | | growth) | growth) | growth) |
| Gel Formulation | 99 | 6 | <3 (No | <3 (No | <3 (No |
| (Example 11) = | | | growth) | growth) | growth) |
| Standard Organism | 150/mL | 90/Ml | 126/mL | 453/mL | 9/mL |
| Control | | | | | |
| Laboratory QC | <1 (No | <1 (No | <1 (No | <1 (No | <1 (No |
| Reagent Water | growth) | growth) | growth) | growth) | growth) |

TABLE 17

| | FORMULATION TESTED | | | |
|---|---|---|---|---|
| | Aspergillus niger | Candida albicans | Escherichia coli | Pseudomonas aeruginosa |
| | (cfu/mL) | (cfu/mL) | (cfu/mL) | (cfu/mL) |
| Day 28 Test Results | Recovery: | Recovery: | Recovery: | Recovery: |
| (@ 1:3) | | | | |
| (Inoculated: Jul. 13, 2011, | | | | |
| 1 mL) | | | | |
| Gel Formulation | 3 | <1 (No | <1 (No | < 1 (No |
| (Example 10) | | growth) | growth) | growth) |
| Gel Formulation | 9 | <1 (No | <1 (No | < 1 (No |
| (Example 11) | | growth) | growth) | growth) |
| Standard Organism | 990 | 42.3 × 10⁶ | 44.4 × 10⁴ | 27.9 × 10¹⁰ |
| Control | | | | |
| Laboratory QC | <1 (No | <1 (No | <1 (No | < 1 (No |
| Reagent Water | growth) | growth) | growth) | growth) |

### Quantitative In Vitro Dentin Tubule Blockage Evaluation of Whitening Gel Applications

An in vitro study was performed to evaluate the tubule occlusion performance of topical whitening solutions over an expedited seven day treatment period as compared to a negative control (tap water). The method, analysis, and results of this study are further set forth below.

Human extracted teeth were collected for this study. The teeth were stored and transported to the study site in a saturated thymol solution. Teeth with root surfaces having no visible damage, malformation or signs of demineralization were selected. Samples (approximately 3 mm diameter) were removed from the tooth roots. Ninety-six (96) specimens were prepared (16 per group). Eight specimens were mounted in an acrylic disk with the dentin surface exposed. The surface to be analyzed was stuck face down on tape. A 1 inch diameter Plexiglas tube ¼ inch deep was placed around the specimens. The tube was filled with acrylic. After curing, the back of the acrylic disk was ground flat. The front (where the specimens were) was then serially polished using 400 grit and 1000 grit paper followed by microcrystalline diamond suspensions of 9, 3 and 1 μm. The disk was as plano-parallel as possible. The dentin blocks were sonicated in deionized water between each grinding/polishing step and before the smear layer removal. To remove the smear layer, the polished specimens were immersed in 1.0% citric acid solution for 30 seconds. The specimens were then sonicated for three 15 minute time periods so they were thoroughly cleaned of acid and debris.

The specimens were assessed under the stereomicroscope at 10× power. To be acceptable for the study, the specimens could not have any obvious cracks or other flaws in the top dentin surface, have an evenly polished high gloss surface at least 3 mm×3 mm around the center of the specimen, and have no visible contamination on the top dentin surface from sticky wax, polish residue or any other material.

When not being used, the dentin blocks were stored at 4° C. under moist conditions in sealed containers to prevent dehydration.

A total of 96 specimens were mounted. Of those, at least 72 dentin specimens (12 per treatment group) were selected and utilized in the in vitro study. To reduce dentin tubule variability, the specimens were examined for baseline tubule occlusion by using a reflective microscope attached to a CCD camera (LECO HARDNESS TESTER). The six most homogeneous specimens on each block were used. The remaining two were identified and not used. This resulted in a total of at least 12 specimens for each group (two discs) being used in the study. If all specimens on a disk were good, all were used.

An indentation (Vicker Indenter) was placed at each end of each row of four specimens so that when positioned correctly the movement of the X-axis moved the optics from one indentation over the center of the four teeth to the second indentation. Two areas of each specimen were evaluated for tubule blockage (according to the scale set forth below) and photographed. While traversing that line, the first scored area and photo was made two rotations of the X axis micrometer (1000μ) from the left edge of each specimen and the second was an additional one rotation of the X axis micrometer (1500μ) from the left edge.

The tooth specimens, assigned to six groups, were treated with embodiments of the oral care formulation disclosed herein as outlined below in Table 18:

TABLE 18

| Group | Formulation for Treatment |
|---|---|
| 1 | Test Solution #1 (Linear polyphosphate |
| | Formulation in Example 1 above) |
| 2 | Test Solution #2 (Cyclic/Cyclic Combination |
| | Polyphosphate Blend Formulation in Example 2 |
| | above) |
| 3 | Test Solution #3 (Cyclic/Linear Combination |
| | Polyphosphate Blend Formulation in Example 3 |
| | above) |
| 4 | Negative Control (tap water) |

The specimens were treated by immersion in their respective whitening gel (or control). For groups 1, 2, & 3, accelerated daily treatments were applied to simulate 30 minute daily usage for seven days. Scoring of the specimens was done after simulated 1, 3 and 7 days of treatment. One day usage consisted of one, 30-minute treatment followed by one hour in pooled human saliva. For group 4, the toothpaste (pea sized sample) was applied undiluted for two minutes with a wet toothbrush (ORAL-B 40, commonly available from retail stores) with light force followed by one hour in human saliva. For group 6, one day usage consisted of application of an accelerator immediately followed by two sequential 10-minute treatments with the gel and then the one hour in pooled human saliva. The specimens were then scored as outlined below. Following scoring, the specimens were then cycled through daily treatments and one hour saliva two additional times and analyzed once again (3 day). The seven day usage consisted of four more daily treatments separated by one hour in pooled human saliva. After treatment cycles representing 1, 3 and 7 days of treatment, the 12 specimens of each group were analyzed for tubule blockage.

Following 30 minutes drying at room temperature, the specimens were observed using the light microscope and post-treatment images were obtained to subjectively determine tubule occlusion over the surface of the specimens. The disks were not removed from the humid environment until 30 minutes prior to scoring and then the scoring was done without interruption. Two areas of each specimen were observed and a general impression of the two areas was given. The following criteria were used for scoring the specimen samples:
1=No occlusion, clean lines around tubules
2=25% or less of the tubules occluded*
3=25-50% of the tubules occluded
4=50-75% of the tubules occluded
5=more than 75% of tubules occluded
6=Cannot see tubules because of coating * occluded is defined as tubules that are no longer clear and/or any amount of deposits are present in the tubules.

The primary outcome variable from the light microscopy analysis is the mean values determined using the subjective scale outlined above. The mean and SEM (Standard Error of Mean) of each parameter for each group was calculated. The summary data are shown in tables 19-21. The raw data was tabulated separately as outlined above. Samples with a higher number showed higher occurrence of tubule occlusion.

Table 19 shows the results from the day 1 analysis. Table 20 shows the same data from the day 3 analyses. Table 21 shows the same data from the day 7 analysis. Based on the results of the laboratory test, the test solutions/formulations of the present invention have some positive effect on occluding dentinal tubules.

The study was conducted and reviewed according to the FDA Monograph on Anticaries Drug Products for Over the Counter Human Use and the FDA Good Laboratory Practices.

TABLE 19
Results After Day 1 of Treatment

| Group | Baseline Score | Post Treatment Score | Change in Score |
|---|---|---|---|
| Tap Water | 1.08 ± 0.06* | **1.08 ± 0.06 | 0.00 ± 0.06 |
| Solution 2 | 1.04 ± 0.04 | 1.04 ± 0.04 | 0.00 ± 0.04 |
| Solution 3 | 1.13 ± 0.07 | 1.00 ± 0.00 | −0.13 ± 0.07 |
| Solution 1 | 1.14 ± 0.07 | 1.14 ± 0.07 | 0.00 ± 0.00 |

*Mean ± SEM (N = 12)

TABLE 20
Results After Day 3 of Treatment

| Group | Baseline Score | Post Treatment Score | Change in Score |
|---|---|---|---|
| Tap Water | 1.08 ± 0.06* | **1.17 ± 0.08 | 0.08 ± 0.08 |
| Solution 2 | 1.04 ± 0.04 | 1.50 ± 0.12 | 0.46 ± 0.12 |
| Solution 3 | 1.14 ± 0.07 | 1.50 ± 0.15 | 0.36 ± 0.15 |
| Solution 1 | 1.13 ± 0.07 | 1.92 ± 0.25 | 0.79 ± 0.26 |

*Mean ± SEM (N = 12)

TABLE 21
Results After Day 7 of Treatment

| Group | Baseline Score | Post Treatment Score | Change in Score |
|---|---|---|---|
| Tap Water | 1.08 ± 0.06* | **1.17 ± 0.08 | 0.08 ± 0.08 |
| Solution 2 | 1.04 ± 0.04 | 1.58 ± 0.16 | 0.54 ± 0.15 |
| Solution 3 | 1.13 ± 0.07 | 1.67 ± 0.18 | 0.54 ± 0.18 |
| Solution 1 | 1.04 ± 0.04 | 1.88 ± 0.15 | 0.83 ± 0.16 |

*Mean ± SEM (N = 12)

There has been provided in accordance with the present invention and the embodiments thereof, an oral care formulation comprising a linear polyphosphate or a polyphosphate blend having a short average effective chain length. The polyphosphate blends may comprise predominantly cyclic combinations of polyphosphate compounds, or “cyclic/cyclic combination polyphosphate blends”, or predominantly linear combinations of polyphosphate compounds, or “cyclic/linear combination polyphosphate blends, There has also been provided in accordance with the present invention and embodiments thereof, a method for making an oral care formulation comprising a linear polyphosphate or polyphosphate blend having a short average effective chain length. There has also been provided in accordance with the present invention and embodiments thereof, a method of applying an oral care formulation comprising a linear polyphosphate or a polyphosphate blend having a short average effective chain length.

While the invention has been described with specific embodiments, many alternatives, modifications and variations will be apparent to those skilled in the art in light of the foregoing description. Accordingly, it is intended to include all such alternatives, modifications and variations set for the within the spirit and scope of the appended claims.

1. An oral care formulation comprising:
a polyphosphate blend having an average effective chain length of from about 3 to about 7.5 repeating units, the polyphosphate blend comprising a cyclic phosphate and a linear polyphosphate; and a carrier base acceptable for oral care formulations.

2. The oral care formulation of claim 1 wherein the ratio of cyclic phosphate to linear polyphosphate is from about 1:4 to about 3:2.

3. The oral care formulation of claim 1 wherein the carrier base further comprises a humectant.

4. The oral care formulation of claim 1 wherein the carrier base further comprises a carbomer.

5. The oral care formulation of claim 1 wherein the carrier base further comprises a pseudoplastic agent.

6. The oral care formulation of claim 1 wherein the carrier base further comprises a buffering agent.

7. The oral care formulation of claim 1 wherein the carrier base further comprises a flavorant.

8. The oral care formulation of claim 1 wherein the carrier base further comprises a sweetener.

9. The oral care formulation of claim 1 wherein the carrier base further comprises a surfactant.

10. The oral care formulation of claim 1 wherein the carrier base further comprises an adhesive.

11. The oral care formulation of claim 1 wherein the carrier base further comprises a lubricant.

12. The oral care formulation of claim 1 further comprising water.

13. The oral care formulation of claim 1 further comprising calcium pyrophosphate.

14. The oral care formulation of claim 1 further comprising an amino acid.

15. The oral care formulation of claim 1 wherein the oral care formulation has a pH of from about 4.0 to about 6.5.

16. An oral care formulation comprising:
from about 4 percent by weight to about 10 percent by weight of a polyphosphate blend having an average effective chain length of from about 3 to about 7.5 repeating units, the polyphosphate blend comprising a cyclic phosphate and a linear polyphosphate; and a carrier base acceptable for oral care formulations.

17. The oral care formulation of claim 16 wherein the carrier base further comprises from about 24 percent by weight to about 45 percent of a humectant.

18. The oral care formulation of claim 16 wherein the carrier base further comprises from about 1 percent by weight to about 5 percent by weight of a carbomer.

19. The oral care formulation of claim 16 wherein the carrier base further comprises from about 0.5 percent by weight to about 9 percent by weight of a buffering agent.

20. The oral care formulation of claim 16 wherein the carrier base further comprises from about 0.1 percent by weight to about 1.0 percent by weight of a pseudoplastic agent.

21. The oral care formulation of claim 16 wherein the carrier base further comprises from about 0.1 percent by weight to about 1.0 percent by weight of a flavorant.

22. The oral care formulation of claim 16 wherein the carrier base further comprises from about 0.05 percent by weight to about 0.2 percent by weight of a sweetener.

23. The oral care formulation of claim 16 wherein the carrier base further comprises from about 35 percent by weight to about 61 percent by weight water.

24. The oral care formulation of claim 16 wherein the carrier base further comprises:
from about 24 percent by weight to about 45 percent of a humectant;
from about 1 percent by weight to about 5 percent by weight of a carbomer;
from about 0.5 percent by weight to about 9 percent by weight of a buffering agent;
from about 0.1 percent by weight to about 1.0 percent by weight of a pseudoplastic agent;
from about 0.1 percent by weight to about 1.0 percent by weight of a flavorant;
from about 0.05 percent by weight to about 0.2 percent by weight of a sweetener; and
from about 35 percent by weight to about 61 percent by weight water;
wherein, the weight percent of each component is selected so that the sum of all components expressed as weight percent is 100.

25. The oral care formulation of claim 16 wherein the formulation has a pH of from about 4.0 to about 6.5.

26. The oral care formulation of claim 16 wherein the ratio of cyclic polyphosphate to linear polyphosphate is from about 1:4 to about 3:2.

27. An oral care formulation comprising:
a polyphosphate blend having an average chain length of from about 3 to about 7.5 repeating units, and a carrier base acceptable for oral care formulations; the carrier base further comprising;
a humectant;
a carbomer;
hydroxide buffering agent;
a pseudoplastic agent;
a flavorant; and
a sweetener.

28. The oral care formulation of claim 27 further comprising an amino acid.

29. The oral care formulation of claim 27 further comprising calcium pyrophosphate.

30. The oral care formulation of claim 27 further comprising water.

31. The oral care formulation of claim 27 wherein the formulation has a pH of from about 4.0 to about 6.5.

32. An oral care formulation comprising:
a polyphosphate blend;
a humectant;
a carbomer;
sodium hydroxide;
xanthan gum;
a flavorant; and
a sweetener.

33. The oral care formulation of claim 32 further comprising calcium pyrophosphate.

34. The oral care formulation of claim 32 further comprising an amino acid.

35. The oral care formulation of claim 32 wherein the polyphosphate blend has an average chain length of from about 3 to about 7.5 repeating units.

36. The oral care formulation of claim 32 wherein the polyphosphate blend is a blend of a cyclic polyphosphate and a linear polyphosphate.

37. The oral care formulation of claim 36 wherein the blend of cyclic polyphosphate and linear polyphosphate has an effective chain length of from about 3 to about 7.5 repeating units.

38. An oral care formulation comprising:
a polyphosphate blend;
an adhesive;
a surfactant;
a buffering agent;
and water.

39. The oral care formulation of claim 38 further comprising an amino acid.

40. The oral care formulation of claim 38 further comprising a flavorant.

41. The oral care formulation of claim 38 further comprising a sweetener.

42. The oral care formulation of claim 38 further comprising calcium pyrophosphate.

43. The oral care formulation of claim 38 wherein the polyphosphate blend has an average chain length of from about 3 to about 7.5 repeating units.

44. The oral care formulation of claim 38 wherein the polyphosphate blend is a blend of a cyclic polyphosphate and a linear polyphosphate.

45. The oral care formulation of claim 38 wherein the blend of cyclic polyphosphate and linear polyphosphate has an effective chain length of from about 3 to about 7.5 repeating units.

46. A method of providing oral care active agents to an oral cavity comprising contacting a subject's dental enamel surfaces and mucosa in the mouth with an oral care formulation, the oral care formulation comprising a polyphosphate blend, the polyphosphate blend selected from the group consisting of a cyclic polyphosphate, a linear polyphosphate, and combinations thereof; and wherein the polyphosphate blend has a short average chain length.

47. A method of preparing an oral care formulation, the oral care formulation comprising a polyphosphate blend, the polyphosphate blend selected from the group consisting of a cyclic polyphosphate, a linear polyphosphate, and combinations thereof; and wherein the polyphosphate blend has a short average chain length, the method comprising:
adding water to the polyphosphate blend in a mixing vessel along with a buffering agent to form a polyphosphate active ingredient mixture;
mixing carbomer, humectant, and pseudoplastic agent to form a carbomer mixture; and
adding the polyphosphate active ingredient mixture to the carbomer mixture.

48. The oral care formulation of claim 38 wherein the polyphosphate is a blend of cyclic polyphosphates.

49. The oral care formulation of claim 1 wherein the polyphosphate blend is further mixed with hydrogen peroxide.

50. An oral care formulation comprising:
a polyphosphate having a pH of less than about 5 pH units; and
a carrier base acceptable for oral care formulations.

51. The oral care formulation of claim 50 wherein the polyphosphate is a linear polyphosphate having an average chain length of from about 3 to about 7.5 repeating units.

52. The oral care formulation of claim 50 wherein the polyphosphate is a blend of a cyclic polyphosphate and a linear polyphosphate.

53. The oral care formulation of claim 52 wherein the blend of cyclic polyphosphate and linear polyphosphate has an effective chain length of from about 3 to about 7.5 repeating units.

54. The oral care formulation of claim 50 wherein the polyphosphate is a blend of cyclic polyphosphates.

55. The oral care formulation of claim 54 wherein the blend of cyclic polyphosphates has an effective chain length of from about 3 to about 7.5 repeating units.

56. The oral care formulation of claim 50 wherein the polyphosphate is further mixed with hydrogen peroxide.

57. An oral care formulation comprising:
from about 4 percent by weight to about 10 percent by weight of a polyphosphate blend having an average effective chain length of from about 3 to about 7.5 repeating units, the polyphosphate blend comprising a blend of cyclic polyphosphates; and a carrier base acceptable for oral care formulations.

58. The oral care formulation of claim 57 wherein the carrier base further comprises from about 24 percent by weight to about 45 percent of a humectant.

59. The oral care formulation of claim 57 wherein the carrier base further comprises from about 1 percent by weight to about 5 percent by weight of a carbomer.

60. The oral care formulation of claim 57 wherein the carrier base further comprises from about 0.5 percent by weight to about 9 percent by weight of a buffering agent.

61. The oral care formulation of claim 57 wherein the carrier base further comprises from about 0.1 percent by weight to about 1.0 percent by weight of a pseudoplastic agent.

62. The oral care formulation of claim 57 wherein the carrier base further comprises from about 0.1 percent by weight to about 1.0 percent by weight of a flavorant.

63. The oral care formulation of claim 57 wherein the carrier base further comprises from about 0.05 percent by weight to about 0.2 percent by weight of a sweetener.

64. The oral care formulation of claim 57 wherein the carrier base further comprises from about 35 percent by weight to about 61 percent by weight water.

65. The oral care formulation of claim 57 wherein the carrier base further comprises:
from about 24 percent by weight to about 45 percent of a humectant;
from about 1 percent by weight to about 5 percent by weight of a carbomer;
from about 0.5 percent by weight to about 9 percent by weight of a buffering agent;
from about 0.1 percent by weight to about 1.0 percent by weight of a pseudoplastic agent;
from about 0.1 percent by weight to about 1.0 percent by weight of a flavorant;
from about 0.05 percent by weight to about 0.2 percent by weight of a sweetener; and
from about 35 percent by weight to about 61 percent by weight water;
wherein, the weigh percent of each component is selected so that the sum of all components expressed as weight percent is 100.

66. The oral care formulation of claim 57 wherein the formulation has a pH of from about 4.0 to about 6.5.

67. The oral care formulation of claim 27 wherein the polyphosphate blend is a blend of a cyclic polyphosphate and a linear polyphosphate.

68. The oral care formulation of claim 27 wherein the polyphosphate blend is a blend of cyclic polyphosphates.

69. An oral care formulation comprising:
a polyphosphate blend having an average effective chain length of from about 3 to about 7.5 repeating units, the polyphosphate blend comprising a blend of a first cyclic phosphate and a second cyclic polyphosphate; and a carrier base acceptable for oral care formulations.

70. The oral care formulation of claim 69 wherein the carrier base further comprises a lubricant.

71. The oral care formulation of claim 69 further comprising an amino acid.

72. The oral care formulation of claim 69 further comprising calcium pyrophosphate.

73. An oral care formulation comprising:
a polyphosphate blend having an average effective chain length of from about 3 to about 7.5 repeating units, the polyphosphate blend comprising a linear polyphosphate; and a carrier base acceptable for oral care formulations.

74. The oral care formulation of claim 73 wherein the carrier base further comprises a lubricant.

75. The oral care formulation of claim 73 further comprising an amino acid.

76. The oral care formulation of claim 73 further comprising calcium pyrophosphate.

77. An oral care formulation comprising:
from about 4 percent by weight to about 10 percent by weight of a polyphosphate blend having an average effective chain length of from about 3 to about 7.5 repeating units, the polyphosphate blend comprising a linear polyphosphate; and a carrier base acceptable for oral care formulations.

78. An oral care formulation comprising:
a polyphosphate having an average chain length of from about 3 to about 7.5 repeating units, and a carrier base acceptable for oral care formulations; the carrier base further comprising;
a humectant;
a carbomer;
sodium hydroxide;
xanthan gum;
a flavorant; and
a sweetener.

79. The oral care formulation of claim 78 wherein the polyphosphate is a blend of a cyclic polyphosphate and a linear polyphosphate.

80. The oral care formulation of claim 78 wherein the polyphosphate is a blend of cyclic polyphosphates.

81. The oral care formulation of claim 78 wherein the polyphosphate is a linear polyphosphate.

82. The oral care formulation of claim 78 further comprising hydrogen peroxide.

83. The oral care formulation of claim 78 further comprising an amino acid.

84. The oral care formulation of claim 78 further comprising calcium pyrophosphate.

85. An oral care formulation comprising:
a polyphosphate having an average chain length of from about 3 to about 7.5 repeating units, and a carrier base acceptable for oral care formulations; the carrier base further comprising;
an adhesive;
a surfactant;
a buffering agent;
and water.

86. The oral care formulation of claim 85 wherein the polyphosphate is a blend of a cyclic polyphosphate and a linear polyphosphate.

87. The oral care formulation of claim 85 wherein the polyphosphate is a blend of cyclic polyphosphates.

88. The oral care formulation of claim 85 wherein the polyphosphate is a linear polyphosphate.

89. The oral care formulation of claim 85 further comprising hydrogen peroxide.

90. The oral care formulation of claim 85 further comprising an amino acid.

91. The oral care formulation of claim 85 further comprising calcium pyrophosphate.
